(19) United States
(12) Patent Application Publication
(10) Pub. No.: US 2025/0172532 A1
(43) Pub. Date: May 29, 2025

(54) METHOD FOR DETECTING FOOD CONTAMINATION USING STABLE ISOTOPE-LABELED STANDARDS

(71) Applicants: Azadeh NASIRI, Tehran (IR); Amirreza NASIRI, Tehran (IR)

(72) Inventors: Azadeh NASIRI, Tehran (IR); Farzad KOBARFARD, Tehran (IR); Amirreza NASIRI, Tehran (IR); Hassan YAZDANPANAH, Tehran (IR); Bahram DARAEI, Tehran (IR)

(73) Assignees: Azadeh NASIRI, Tehran (IR); Amirreza NASIRI, Tehran (IR)

(21) Appl. No.: 19/039,786

(22) Filed: Jan. 29, 2025

Related U.S. Application Data
(63) Continuation-in-part of application No. PCT/IB2023/056891, filed on Jul. 3, 2023.
(60) Provisional application No. 63/395,792, filed on Aug. 6, 2022.

(51) Int. Cl. G01N 33/02; G01N 30/02; G01N 30/06; G01N 30/72; G01N 30/86; G01N 30/88

(52) U.S. Cl. CPC G01N 33/02; G01N 30/7233; G01N 30/8679; G01N 30/88; G01N 2030/027; G01N 2030/062; G01N 2030/8872

(57) ABSTRACT

A method for detecting contamination of a food sample with a target analyte by detecting whether the target analyte exceeds a maximum residue limit (MRL) of the target analyte in the food sample considering matrix effects specific to the food sample and essentially without requiring construction of a calibration curve. The method includes spiking the food sample with a stable isotope-labeled analyte analogous to the target analyte at a concentration of L_{eq} equivalent to the MRL of the target analyte. L_{eq} is defined by the following operation,

$${L}_{eq} = {W}_{N} \times \frac{MW ( L )}{MW ( N )} \times \frac{purity ( N )}{purity ( L )} .$$

The method further includes extracting the stable isotope-labeled analyte and the target analyte from the spiked food sample, generating an ion chromatogram including a first peak representing the stable isotope-labeled analyte and a second peak representing the target analyte, and calculating and comparing respective areas under the first peak and the second peak.

### CROSS-REFERENCE TO RELATED APPLICATION

This application claims the benefit of priority from U.S. Provisional Patent Application Ser. No. 63/395,792, filed on Aug. 6, 2022, entitled “STABLE ISOTOPE LABELLED ANALYTES WITH A SURROGATE ANALYTE APPROACH IN FOOD SCREENING FOR CHEMICAL CONTAMINANTS” which is incorporated herein by reference in its entirety.

### TECHNICAL FIELD

The present disclosure is generally related to an exemplary method for detecting contamination of a food sample with one or more chemical residues/contaminants using stable isotope-labeled analytes (internal standards), without needing to create calibration curves.

### BACKGROUND OF THE INVENTION

As foods are being produced and distributed in large scales in today's marketplace, food quality and safety has become an increasing concern for governments, producers, and consumers. Chemical contaminants in food may be defined as any chemical substance that may be added to food, unintentionally (e.g., from environmental sources) or intentionally (food adulteration), and may include residues that enter food chain from environmental sources, residues generated from using pesticides and veterinary medicines, natural toxins, residues formed during food processing, accidental contamination at point sources, etc. Many governments have established regulatory limits, known as maximum residue limits (MRLs), as part of a regulatory approval process for active substances/ingredients and product use authorization. An MRL defines a maximum concentration of a residue that is legally allowed or recognized as acceptable in a food product. A food sample may be considered as contaminated when concentration of a contaminant in the food sample exceeds MRL.

Using conventional techniques, such as liquid chromatography-mass spectrometry (LC-MS), to screen food products for the presence of food contaminants at MRL level may require conducting a large number of standard tests and creating calibration curves for different food matrices. Generating calibration curves to set up screening assays for each chemical contaminant and each food matrix may be significantly expensive and time-consuming. Thus, there is a need to develop sensitive and specific screening methods that are independent of generating calibration curves and are capable of minimizing matrix effect, instrumental variations, and operational variations/errors.

### SUMMARY OF THE INVENTION

This summary is intended to provide an overview of the subject matter of the present disclosure, and is not intended to identify essential elements or key elements of the subject matter, nor is it intended to be used to determine the scope of the claimed implementations. Its sole purpose is to present some concepts of one or more exemplary aspects in a simplified form as a prelude to the more detailed description that is presented later. The proper scope of the present disclosure may be ascertained from the claims set forth below in view of the detailed description below and the drawings.

One or more exemplary embodiments describe an exemplary method for detecting contamination of an exemplary food sample with an exemplary target analyte. In an exemplary embodiment, an exemplary method may include spiking an exemplary food sample with an exemplary stable isotope-labeled analyte analogous to an exemplary target analyte, extracting an exemplary stable isotope-labeled analyte and an exemplary target analyte from an exemplary spiked food sample, generating an ion chromatogram (IC) by introducing an exemplary extracted stable isotope-labeled and target analyte into a liquid chromatography-mass spectrometer (LC-MS) where an exemplary IC may include a first peak representing an exemplary stable isotope-labeled analyte and a second peak representing an exemplary target analyte, calculating an area under an exemplary first peak and an area under an exemplary second peak utilizing one or more processors, and detecting contamination of an exemplary food sample with an exemplary target analyte by comparing an exemplary area under an exemplary first peak with an exemplary area under an exemplary second peak utilizing one or more processors.

In an exemplary embodiment, spiking an exemplary food sample with an exemplary stable isotope-labeled analyte analogous to an exemplary target analyte may include spiking an exemplary food sample with an exemplary stable isotope-labeled analyte at a level equivalent to a maximum residue limit (MRL) of the target analyte being permissible to be present in the food sample. In an exemplary embodiment, spiking an exemplary food sample with an exemplary stable isotope-labeled analyte analogous to an exemplary target analyte may include adding an amount of an exemplary stable isotope-labeled analyte with a final concentration to an exemplary food sample equivalent to an exemplary MRL of an exemplary target analyte.

In an exemplary embodiment, spiking an exemplary food sample with an exemplary stable isotope-labeled analyte analogous to an exemplary target analyte may include adding an exemplary stable isotope-labeled analyte with a final concentration of Leq to an exemplary food sample. In an exemplary embodiment, Leq may be defined by following operation:

$$Leq = WN \times \frac{MW ( L )}{MW ( N )} \times \frac{purity ( N )}{purity ( L )} \qquad Equation ( 1 )$$

In an exemplary embodiment, with regards to Equation 1, Leq may be an exemplary final concentration of an exemplary stable isotope-labeled analyte in an exemplary spiked food sample, WN may be a maximum residue limit (MRL) of an exemplary target analyte, MW(L) may be molecular weight of an exemplary stable isotope-labeled analyte, MW(N) may be molecular weight of an exemplary target analyte, purity(N) may be an exemplary percentage purity (w/w) of an exemplary target analyte, and purity(L) may be an exemplary percentage purity (w/w) of an exemplary stable isotope-labeled analyte.

In an exemplary embodiment, WN may include MRL of an exemplary target analyte in parts per billion (ppb) of an exemplary food sample. In an exemplary embodiment, WN may include MRL of an exemplary target analyte in parts per million (ppm) of an exemplary food sample.

In an exemplary embodiment, spiking an exemplary food sample with an exemplary stable isotope-labeled analyte at an exemplary level equivalent to an exemplary MRL of an exemplary target analyte may include selecting an exemplary stable isotope-labeled analyte, calculating amount of an exemplary Leq utilizing one or more processors, and adding an exemplary stable isotope-labeled analyte with an exemplary final concentration equal to Leq to an exemplary food sample.

In an exemplary embodiment, extracting an exemplary stable isotope-labeled analyte and an exemplary target analyte from an exemplary spiked food sample may include extracting an exemplary stable isotope-labeled analyte and an exemplary target analyte from an exemplary spiked food sample through at least one of a solid-liquid extraction process, a liquid-liquid extraction process, a solid-phase extraction process, and combinations thereof.

In an exemplary embodiment, detecting an exemplary contamination of an exemplary food sample with an exemplary target analyte may include detecting contamination of an exemplary food sample with a chemical target analyte. In an exemplary embodiment, detecting an exemplary contamination of an exemplary food sample with an exemplary target analyte may include detecting contamination of an exemplary food sample with at least one of a pesticide compound, an antibiotic compound, a drug, a toxin, a heavy metal, a persistent Organic Pollutant (POP), Brominated Fire Retardants (BFR), a phthalate, a dioxins, a halogenated compound, a hormone, and combinations thereof. In an exemplary embodiment, detecting an exemplary contamination of an exemplary food sample with an exemplary target analyte may include detecting contamination of an exemplary food sample with at least one of Dinitrocarbanilide, Enrofloxacin-HCl, Ciprofloxacin, Marbofloxacin, Danofloxacin-mesylate, Flumequine, Sulfamethazine, and Spiramycin I, and combinations thereof.

In an exemplary embodiment, detecting an exemplary contamination of an exemplary food sample with an exemplary target analyte may include detecting an exemplary contamination of a sample of at least one of baked goods, baking mixes, flours, coffee, tea, milk, breakfast cereals, fats and oils, condiments, relishes, fresh fruits, fruit juices, herbs, spices, fresh vegetables, meat products, jams, jellies, snack foods, soups, sugars, water, rice, corn, wheat, coffee grounds, tea leaves, food additives, chemicals used in food production, dyes, color additives, vitamins, dietary supplements, medicines, cosmetics, and combinations thereof.

In an exemplary embodiment, generating an exemplary IC may include injecting an exemplary extracted stable isotope-labeled and target analyte into an inlet chamber of an exemplary LC-MS using a syringe pump and plotting an exemplary IC associated with an exemplary injected extracted stable isotope-labeled and target analyte utilizing one or more processors. In an exemplary embodiment, an exemplary IC may include peak intensities of an exemplary injected extracted stable isotope-labeled and target analyte versus retention time (RT) of an exemplary injected extracted stable isotope-labeled and target analyte passing through a chromatography column of the LC-MS.

In an exemplary embodiment, comparing an exemplary area under an exemplary first peak with an exemplary area under the second peak may include calculating a ratio of target analyte peak area to stable isotope-labeled analyte peak area by dividing an exemplary area under an exemplary second peak to an exemplary area under an exemplary first peak utilizing one or more processors and detecting contamination status of an exemplary food sample utilizing one or more processors. In an exemplary embodiment, detecting contamination status of an exemplary food sample may include one of detecting that an exemplary food sample is contaminated with an exemplary target analyte if an exemplary ratio of an exemplary target analyte peak area to an exemplary stable isotope-labeled analyte peak area is more than 1 (>1) or detecting that an exemplary food sample is not contaminated with an exemplary target analyte if an exemplary ratio of an exemplary target analyte peak area to an exemplary stable isotope-labeled analyte peak area is less than 1 (<1).

In an exemplary embodiment, an exemplary method may further include removing whole of food product associated with an exemplary food sample from distribution in markets an exemplary food sample is detected to be contaminated with an exemplary target analyte. In an exemplary embodiment, an exemplary food sample may be sampled from the whole of the food product.

In an exemplary embodiment, an exemplary method may further include preparing a detection mixture. In an exemplary embodiment, an exemplary detection mixture may include a plurality of stable isotope-labeled analytes analogous to a plurality of target analytes. In an exemplary embodiment, a concentration of each stable isotope-labeled analyte of an exemplary plurality of stable isotope-labeled analytes in an exemplary detection mixture may correspond to a respective MRL of each target analyte of an exemplary plurality of target analytes. In an exemplary embodiment, an exemplary plurality of stable isotope-labeled analytes may include an exemplary stable isotope-labeled analyte and an exemplary plurality of target analytes may include an exemplary target analyte to be detected in an exemplary food sample. In an exemplary embodiment, spiking an exemplary food sample with an exemplary stable isotope-labeled analyte may include adding an exemplary detection mixture to an exemplary food sample.

In an exemplary embodiment, preparing an exemplary detection mixture may include calculating an exemplary concentration of each stable isotope-labeled analyte of an exemplary plurality of stable isotope-labeled analytes equal to an exemplary Leq of each respective target analyte of an exemplary plurality of target analytes and mixing an exemplary plurality of stable isotope-labeled analytes at exemplary calculated concentrations together.

In an exemplary embodiment, mixing an exemplary plurality of stable isotope-labeled analytes at exemplary calculated concentrations together may include dissolving an exemplary plurality of stable isotope-labeled analytes at exemplary calculated concentrations in at least one of an aqueous solvent, an organic solvent, and combinations thereof. In an exemplary embodiment, mixing an exemplary plurality of stable isotope-labeled analytes at exemplary respective calculated concentrations together may include dissolving an exemplary plurality of stable isotope-labeled analytes at exemplary respective calculated concentrations in at least one of water, an alcohol, acetonitrile, and combinations thereof. In an exemplary embodiment, preparing an exemplary detection mixture may further include removing at least one of an exemplary aqueous solvent, an exemplary organic solvent, and combinations thereof from an exemplary plurality of stable isotope-labeled analytes dissolved therein.

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF DRAWINGS

The novel features which are believed to be characteristic of the present disclosure, as to its structure, organization, use and method of operation, together with further objectives and advantages thereof, will be better understood from the following drawings in which an exemplary embodiment will now be illustrated by way of example. It is expressly understood, however, that the drawings are for the purpose of illustration and description only and are not intended as a definition of the limits of the present disclosure. Exemplary embodiments will now be described by way of example in association with the accompanying drawings in which:

FIG. 1 illustrates flowchart of an exemplary method for detecting contamination of an exemplary food sample with an exemplary target analyte based on an exemplary MRL of an exemplary target analyte, consistent with one or more exemplary embodiments of the present disclosure;

FIG. 2 illustrates an exemplary ion chromatogram (IC) plot of an exemplary extracted dinitrocarbanilide (DNC) and deuterium-labeled DNC (DNC-d8) generated by an exemplary liquid chromatography-mass spectrometer (LC-MS), consistent with one or more exemplary embodiments of the present disclosure;

FIG. 3 illustrates an example computer system in which an embodiment of the present invention, or portions thereof, may be implemented as computer-readable code, consistent with exemplary embodiments of the present disclosure; and

FIG. 4 illustrates an exemplary IC plot obtained from LC-MS analysis of an exemplary milk sample spiked with an exemplary mixture of target quinolones (enrofloxacin, ciprofloxacin, danofloxacin, marbofloxacin and flumequine) and an exemplary mixture of stable isotope-labeled quinolones analogous to target quinolones, consistent with one or more exemplary embodiments of the present disclosure.

### DETAILED DESCRIPTION OF THE INVENTION

In the following detailed description, numerous specific details are set forth by way of examples to provide a thorough understanding of the relevant teachings related to the exemplary embodiments. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in one or more exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be plain to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Liquid chromatography-mass spectrometry (LC-MS) has revolutionized food safety analysis by providing sensitive and selective data relating to the structure and possible identity of target compounds. Notwithstanding, one of the technical challenges with LC-MS may have been instrumental variations, matrix effect (i.e., effect of sample matrix), and operational variations that may variate LC-MS responses from test to test. As used herein, “matrix effect” may refer to a phenomenon in which matrix components may be co-eluted with target analyte and interfere with ionization process of target analyte in mass spectrometry (MS). Using internal standards, such as stable isotope-labeled analogues of a target analyte, in LC-MS has been demonstrated to minimize matrix effect by correcting variability in dilutions, degradation, evaporation, adsorption, recovery, derivatization, and instrumental parameters such as injection volume. To minimize effects of instrumental variations, operational variations, and sample matrix, conventional methods may use calibration/standard curves in which y-axis represents ratio of target analyte signal intensity to internal standard signal intensity (instead of absolute signal intensity values of target analyte) and x-axis represents analyte concentration. Commonly, national/international food and agriculture organizations group different food matrices into a number of commodities/categories (e.g., 5-10). As setting up calibration curves/tests is a time-consuming procedure, food safety laboratories are obligated to set up calibration curves for at least 2 different food products of each commodity and generalize exemplary obtained calibration curves to other food products in each commodity, which may increase the risk of acquiring erroneous analytical results due to not considering matrix effect of each food product, separately. Herein, a method is disclosed for screening food and agricultural products for contaminants. An exemplary method may include use of stable isotope-labeled contaminants as surrogate analytes for target contaminants, where an exemplary method may enable determination of contamination levels exceeding maximum residue limits (MRLs) without requiring construction of a calibration curve, and may provide compensation to mitigate matrix effects specific to each food matrix being analyzed. Herein, each target contaminant may be measured using its corresponding stable isotope-labeled molecule substituted in a target food sample to be analyzed; thereby, matrix effect of each food product may be considered in all measurements as well as not requiring forming a calibration curve. Hence, conducting an exemplary method for detection of contaminants in food and agricultural products disclosed herein may overcome the above-mentioned problems and errors of common methods due to a requirement to construct a calibration curve and ignoring matrix effects.

Disclosed herein is an exemplary method for detecting contamination of an exemplary food sample with an exemplary target analyte (using LC-MS), taking an exemplary maximum residue limit (MRL) of an exemplary target analyte as a reference value. “Maximum residue limit (MRL),” also known as “legal limit,” “regulatory limit,” “national residue limit,” and “tolerance,” may refer to a maximum concentration of a residue that is legally allowed or recognized as acceptable in a food product. In many countries, MRLs may be established as part of a regulatory approval process for active substances/ingredients and product use authorization. “Target analyte” may refer to a substance/molecule (e.g., a food contaminant) that is intended to be detected or quantified in a sample. In one or more exemplary embodiments, an exemplary target analyte may include, but is not limited to, a pesticide compound, an antibiotic compound, a drug, a toxin, a heavy metal, a persistent Organic Pollutant (POP), Brominated Fire Retardants (BFR), a phthalate, a dioxins, halogenated compounds, hormones, and/or any food contaminant that has an analogous isotope-labeled molecule. “Food sample” may refer to a food product selected from an exemplary food commodity/category including, but not limited to, baked goods and baking mixes (including flours), coffee and tea, breakfast cereals, milk, fats and oils, condiments and relishes, fresh fruits and fruit juices, herbs and spices, fresh vegetables, meat products, jams and jellies, snack foods, soups, sugars, water, rice, corn, wheat, coffee grounds and tea leaves, food additives, certain chemicals used in food production, dyes or color additives, vitamins, dietary supplements, medicines, and cosmetics, etc.

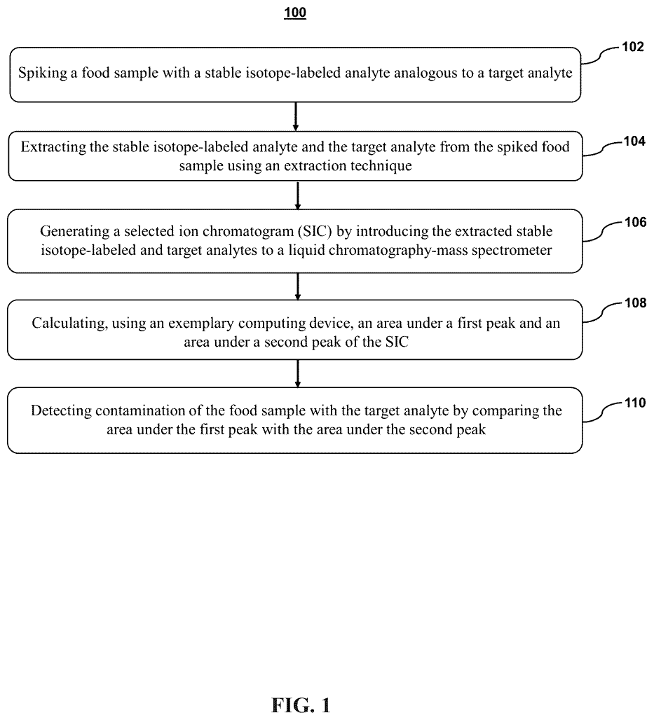

Referring to the figures, FIG. 1 illustrates flowchart of exemplary method 100 for detecting contamination of an exemplary food sample with an exemplary target analyte based on an exemplary MRL of an exemplary target analyte, consistent with one or more exemplary embodiments of the present disclosure. In an exemplary embodiment, exemplary method 100 may include: spiking an exemplary food sample with an exemplary stable isotope-labeled analyte analogous to an exemplary target analyte (step 102); extracting an exemplary stable isotope-labeled analyte and an exemplary target analyte from an exemplary spiked food sample using an exemplary extraction technique (step 104); generating an exemplary ion chromatogram (IC) by introducing exemplary extracted stable isotope-labeled and target analyte to an exemplary LC-MS (step 106); calculating, using an exemplary computing device, an exemplary area under an exemplary first peak and an exemplary area under an exemplary second peak of an exemplary IC (step 108); and detecting contamination of an exemplary food sample with an exemplary target analyte by comparing an exemplary area under an exemplary first peak with an exemplary area under an exemplary second peak (step 110).

In an exemplary embodiment, detecting an exemplary contamination of an exemplary food sample with an exemplary target analyte may include detecting contamination of an exemplary food sample with a chemical target analyte. In an exemplary embodiment, detecting an exemplary contamination of an exemplary food sample with an exemplary target analyte may include detecting contamination of an exemplary food sample with an antibiotic compound. In an exemplary embodiment, detecting an exemplary contamination of an exemplary food sample with an exemplary target analyte may include detecting contamination of an exemplary food sample with quinolones. In an exemplary embodiment, detecting an exemplary contamination of an exemplary food sample with an exemplary target analyte may include detecting contamination of an exemplary food sample with at least one of Dinitrocarbanilide, Enrofloxacin-HCl, Ciprofloxacin, Marbofloxacin, Danofloxacin-mesylate, Flumequine, Sulfamethazine, and Spiramycin I, and combinations thereof. In an exemplary embodiment, an exemplary food sample may include chicken meat or milk.

In further detail with respect to step 102, step 102 may include spiking an exemplary food sample with an exemplary stable isotope-labeled analyte analogous to an exemplary target analyte. In an exemplary embodiment, “spiking” may refer to adding a known amount/concentration of a known substance to a sample, that is, adding exemplary stable isotope-labeled analyte to an exemplary food sample. In an exemplary embodiment, “stable isotope-labeled analyte,” also known as “stable isotope-labeled standard” or “stable isotope-labeled internal standard,” may refer to compounds in which one or more atoms (e.g., hydrogen (1H), Carbon (6C), and/or Nitrogen (7N), etc.) are replaced by their analogous stable (non-radioactive) isotopes, such as deuterium (2H or D), 13C, 15N, etc. Every chemical element in periodic table may have one or more isotopes with a same atomic number. Physicochemical behavior of an isotope-labeled molecule may be expected to be very similar to its unlabeled version. Higher molecular weight afforded by isotopes makes stable isotope-labeled analytes ideal internal standards for chromatographic methods coupled to mass spectroscopic detection. Using stable isotope-labeled analytes as internal standards may reduce matrix effect and render reproducible and accurate recoveries in LC-MS assays.

In an exemplary embodiment, spiking an exemplary food sample with an exemplary stable isotope-labeled analyte analogous to an exemplary target analyte may include spiking an exemplary food sample with an exemplary stable isotope-labeled analyte having an exemplary final concentration equal to Leq (i.e., adding an exemplary stable isotope-labeled analyte with an exemplary final concentration of Leq to an exemplary food sample). In an exemplary embodiment, adding an exemplary stable isotope-labeled analyte with an exemplary final concentration of Leq to an exemplary food sample may include adding (e.g., using a spatula, a graduated cylinder, a pipet, etc.) and mixing (e.g., using a spatula, stirring rod, etc.) an exemplary stable isotope-labeled analyte analogous to an exemplary target analyte (e.g., in liquid or powder forms), with an exemplary final concentration of Leq, to an exemplary food sample, in a laboratory container (e.g., beakers, tins, flasks, bottles, buckets, basins, bowls, vials, tubes, barrels, cannisters, etc.). In an exemplary embodiment, Leq may be defined by the following operation:

$${L}_{eq} = {W}_{N} \times \frac{MW ( L )}{MW ( N )} \times \frac{purity ( N )}{purity ( L )} \qquad Equation ( 1 )$$

In an exemplary embodiment, with respect to Equation 1 above, Leq may denote an exemplary final concentration of an exemplary stable isotope-labeled analyte in an exemplary spiked food sample, WN may denote an exemplary MRL of an exemplary target analyte (e.g., in parts per million or parts per billion), MW(L) may denote molecular weight of an exemplary stable isotope-labeled analyte, MW(N) may denote molecular weight of an exemplary target analyte, purity(N) may denote percentage purity (w/w) of an exemplary target analyte, and purity(L) may denote percentage purity (w/w) of an exemplary stable isotope-labeled analyte. In an exemplary embodiment, “percentage purity” may refer to mass of a pure compound divided by total mass of a sample material containing the pure compound multiplied by 100. In an exemplary embodiment, Equation 1 may help to obtain a final concentration (Leq) of an exemplary stable isotope-labeled analyte that may be equivalent to MRL of an exemplary target analyte (i.e., may result in a same mass spectrometry (MS) ion plot as ion plot of an exemplary target analyte with a concentration equal to MRL). In other words, introducing an exemplary stable isotope-labeled analyte with a final concentration of Leq to an exemplary MS may result in a signal with a same intensity and peak area count as the signal of an exemplary target molecule with a concentration equal to MRL. Thereby, an exemplary MS signal of an exemplary stable isotope-labeled analyte, spiked into an exemplary food sample, may be used as a reference to compare concentration level of an exemplary target analyte to MRL of an exemplary target analyte (set forth in further detail in steps 106-110). For example, an exemplary method similar to exemplary method 100 may be used to detect dinitrocarbanilide (DNC) in chicken meat. DNC is an antiprotozoal drug used to control coccidiosis in poultry. The Joint Expert Committee on Food Additives (JECFA) of the Codex Alimentarius Commission has evaluated residue depletion data and elaborated an MRL of 200 parts per billion (ppb or ng/g) in poultry liver samples. In an exemplary embodiment, an exemplary method for detecting DNC in an exemplary food sample may include an exemplary procedure similar to step 102. In an exemplary embodiment, an exemplary procedure similar to step 102 may include adding and mixing (e.g., using a spatula, stirring rod, blender, etc.) an exemplary deuterium-labeled DNC (DNC-d8 (C13D8H2N4O5); e.g., in form of powder), with a final concentration (Leq) calculated by Equation 1, to an exemplary blended chicken meat containing an unknown concentration of DNC, in an exemplary laboratory container. In an exemplary embodiment, calculated Leq for DNC-d8 based on Equation 1 may equal to about 205.5 ppb (WN=200 ppb, MW(L)=310.29, MW(N)=302.24, purity(N)=100%, purity(L)=99.9%).

In further detail with respect to step 104, step 104 may include extracting an exemplary stable isotope-labeled analyte and an exemplary target analyte from an exemplary spiked food sample using an exemplary extraction technique. In an exemplary embodiment, extracting an exemplary stable isotope-labeled analyte and an exemplary target analyte from an exemplary spiked food sample may include extracting an exemplary stable isotope-labeled analyte and an exemplary target analyte from an exemplary spiked food sample using an exemplary extraction technique including, but not limited to, solid-liquid extraction, liquid-liquid extraction, and solid-phase extraction techniques. In an exemplary embodiment, “liquid-liquid extraction,” also known as solvent extraction, may refer to exchange of one or more compounds between two solvents that are immiscible or partially miscible. Liquid-liquid extraction separates chemicals from one solution to another solution based on the different solubility of a solute in two solvents. In an exemplary embodiment, “solid-liquid extraction” may refer to partitioning of analytes between two phases, i.e., a matrix and an extractant. Solid-liquid extraction process may be regulated by three essential mechanisms including: penetration of extractant in a solid matrix, diffusion of analytes to an outer space, and dissolving analytes in an extractant. A common form of solid-liquid extraction may comprise combining a solid with a solvent in which an analyte is soluble. Through agitation, an analyte may be separated into liquid phase that may then be separated from solid through filtration. Choosing a solvent for solid-liquid extraction may be based on solubility of a target analyte. In an exemplary embodiment, “solid-phase extraction” may refer to a sample preparation technique that isolates one or more analytes from a liquid sample by extracting, partitioning, and/or adsorbing onto a solid stationary phase. Solid-phase extraction techniques may be commonly used to remove interfering compounds from a sample; however, solid-phase extraction may also be used to enrich/concentrate analytes of interest in a sample. Solid-phase extraction entails a solid phase material that is capable of retaining interfering substances, while solvents elute from sample to further be analyzed. Solid-phase extraction techniques may fall into several categories, including normal phase, reversed phase, ion exchange (anion/cation), and mixed-mode phases (that include properties of more than one type of solid-phase extraction material).

In an exemplary embodiment, extracting an exemplary stable isotope-labeled analyte and an exemplary target analyte from an exemplary spiked food sample using an exemplary extraction technique may include extracting an exemplary stable isotope-labeled analyte and an exemplary target analyte from an exemplary spiked food sample using an exemplary liquid-liquid extraction technique (solvent extraction technique). In an exemplary embodiment an exemplary liquid-liquid extraction technique may include a plurality of stages of penetration of an exemplary solvent into an exemplary food sample (that may be an exemplary solid matrix), dissolving of exemplary solutes (i.e., an exemplary target analyte and an exemplary stable isotope-labeled analyte) into an exemplary solvent, diffusion of exemplary solutes out of an exemplary food sample, and collection of exemplary extracted solutes. In an exemplary embodiment, an exemplary solvent may be an exemplary aliphatic/organic solvent (a solvent including organic molecules with 1-12 carbons). An exemplary aliphatic/organic solvent may include acetonitrile, hexanes, toluene, dichloromethane, diethyl ether, etc. It is appreciated by a person skilled in the art that an ideal extraction technique/procedure may be chosen based on the type of an exemplary food sample matrix and the type of an exemplary food contaminant (i.e., an exemplary target analyte) that are intended to be analyzed. For example, an exemplary procedure similar to step 104 may be used to extract DNC and DNC-d8 from an exemplary spiked food sample (containing an unknown amount of DNC and about 205.5 ppb or ng/g of DNC-d8). In an exemplary embodiment, extracting DNC and DNC-d8 from an exemplary blended chicken meat (i.e., spiked chicken meat) may include: adding about 2 mL acetonitrile per 1 g of an exemplary blended chicken meat (e.g., 10 mL acetonitrile for 5 g blended chicken meat); stirring an exemplary mixture of spiked chicken meat and acetonitrile using a spatula, stirring rod, stirrer, and/or blender; centrifuging an exemplary stirred mixture at about 3000 rpm for a time duration of about 10 minutes; collecting an exemplary supernatant formed after centrifugation using a sampler followed by transferring an exemplary collected supernatant to an exemplary laboratory container (e.g., a falcon, etc.); and evaporating acetonitrile from an exemplary collected supernatant under a stream of nitrogen gas in an exemplary nitrogen evaporator. In an exemplary embodiment, an exemplary acetonitrile-based extraction procedure described above may be repeated once or more on an exemplary pellet formed after centrifugation (followed by adding an exemplary obtained supernatant to previous supernatants) to extract a maximum amount of DNC and DNC-d8.

In further detail with respect to step 106, step 106 may include generating an exemplary IC by introducing exemplary extracted stable isotope-labeled and target analyte into an exemplary LC-MS. “Liquid chromatography-mass spectrometry (LC-MS)” may refer to an analytical system in which a liquid chromatography (LC) system is coupled to an MS through an interface. When a column eluent eluting from LC flows into an interface, solvent of eluent is evaporated by applying heat (that is applied automatically in MS) and analyte molecules are vaporized and ionized. Vaporization that is operated by MS is a crucial step because MS is only capable of detecting and quantifying gas phase ions. The interface of MS is known as atmospheric pressure ionization (API) source and may ionize analytes to molecular/analyte ions at atmospheric pressure. An ionization source in LC-MS may include, but is not limited to, electrospray ionization (ESI) and atmospheric pressure chemical ionization (APCI). Molecular ions may then be drawn into MS where they are subjected to magnetic and/or electric fields. Flight paths of molecular ions may be altered by changing applied fields such that ions may become separated from one another based on their mass-to-charge (m/z) values. After separation of molecular ions, ions may be collected and detected by a variety of mass detectors, such as electron-multiplier. When separated molecular ions strike a surface of electron-multiplier, secondary electrons may be released. Secondary electrons may then be multiplied by being cascaded through a series of electron-multipliers that may result in an amplification of current. Generated current may be quantified and correlated to ion concentrations in MS at any given instant in time. Abundances of molecular ions measured by LC-MS may be plotted as a total ion chromatogram (TIC). TIC plot may display peak intensities of analyte/molecular ions versus their retention time (RT; the amount of time spent by a compound to pass through a chromatography column). Each point in TIC may be associated with a mass spectrum that depicts ion abundances versus quantified m/z values.

Furthermore, in an exemplary embodiment, introducing exemplary extracted stable isotope-labeled and target analyte into an exemplary LC-MS may include injecting (using a syringe pump) exemplary extracted stable isotope-labeled and target analyte into an inlet chamber of an exemplary LC-MS. In an exemplary embodiment, generating an exemplary IC by introducing exemplary extracted stable isotope-labeled and target analyte into an exemplary LC-MS may include generating an exemplary TIC and extracting, from an exemplary TIC, an exemplary IC by introducing exemplary extracted stable isotope-labeled and target analyte to an exemplary LC-MS. In an exemplary embodiment, “ion chromatogram (IC)” may refer to a plot of ion current versus RT obtained from LC-MS. IC may provide for monitoring a small mass window within TIC that represents detection of a very specific m/z ratio or an ion transition. IC may be extracted from TIC by tracing mass slices with several continuous scans by advanced pattern recognition and/or video processing-based object tracing or by binning data points in two-dimensional space (scan number and m/z) into a centroid mass with a specific tolerance.

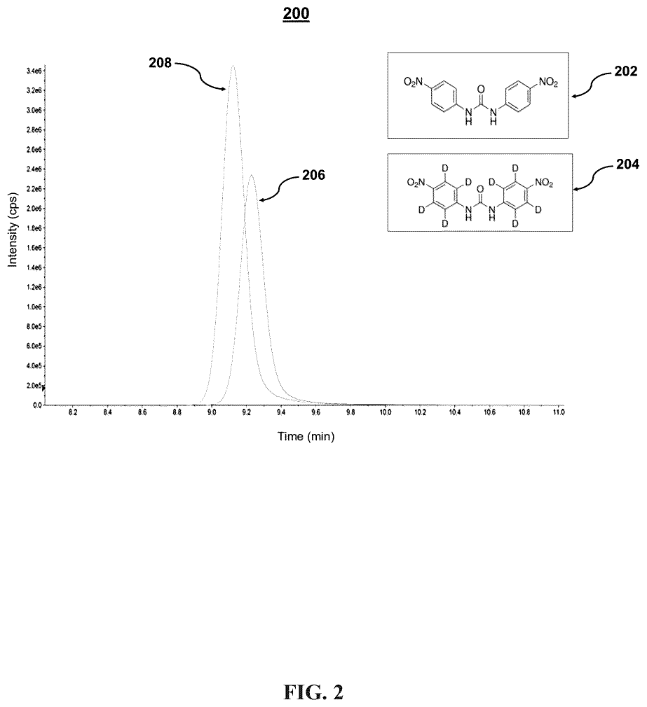

In an exemplary embodiment, an exemplary IC may include an exemplary first peak representing an exemplary stable isotope-labeled analyte and an exemplary second peak representing an exemplary target analyte. For example, an exemplary procedure similar to step 106 may be used to generate an exemplary IC plot for exemplary extracted DNC and DNC-d8 (extracted using an exemplary procedure similar to step 104) by LC-MS analysis. In an exemplary embodiment, an exemplary IC plot generated by injecting (using a syringe pump) exemplary extracted DNC and DNC-d8 into an inlet source of an exemplary LC-MS (based on an exemplary procedure similar to step 106) is illustrated in FIG. 2. FIG. 2 illustrates exemplary IC plot 200 of exemplary extracted DNC 202 and DNC-d8 204 generated by an exemplary LC-MS, consistent with one or more exemplary embodiments of the present disclosure. In further detail with respect to FIG. 2, peak 206 represents intensity of DNC ions and peak 208 represents intensity of DNC-d8 ions in an exemplary sample (obtained from an exemplary procedure similar to step 104) introduced into an exemplary LC-MS. In an exemplary embodiment, an exemplary TIC and/or IC may be plotted and/or selected using one or more processors. In an exemplary embodiment, an exemplary TIC and/or IC may be plotted and/or selected using a computing device.

In further detail with respect to step 108, step 108 may include calculating, using an exemplary computing device, an exemplary area under an exemplary first peak and an exemplary area under an exemplary second peak of an exemplary IC. In an exemplary embodiment, “area under a peak” or “peak area count” may refer to a measure of the concentration/amount of a compound by integrating and calculating an exemplary area enclosed by a peak in a plot. In an exemplary embodiment, peak area count may be integrated and calculated automatically by an exemplary computer data station. In an exemplary embodiment, “peak integration” may refer to at least one mathematical algorithm and/or operation for determining a peak area enclosed by a peak in a chromatogram plot (e.g., TIC and/or IC). In particular, peak integration may include identification and/or measurement of peak/curve characteristics. In an exemplary embodiment, peak integration may include at least one or a combination of peak finding/detection/identification, peak fitting, peak evaluation, determining an upper peak boundary and/or a lower peak boundary, determining peak background, and/or determining peak basis line. Peak integration may be used to calculate at least one of retention time, peak area, peak width, and peak height. In an exemplary embodiment, step 108 may be performed using exemplary one or more processors. In an exemplary embodiment, peak detection/identification, peak integration, and/or peak area count calculation may be performed automatically, i.e., without manual calculations (e.g., by using an exemplary computing device). In an exemplary embodiment, “identifying/detecting a peak” may refer to any measurement that may determine at least one parameter of a peak in a chromatogram plot. In an exemplary embodiment, peak identification/detection may include identifying an upper peak boundary and/or a lower peak boundary, peak purity, and/or determining a ratio of target analyte peak area to internal standard peak area (i.e., stable isotope-labeled analyte peak area), known as peak area ratio. In an exemplary embodiment, an exemplary procedure similar to step 108 may be implemented to calculate area under exemplary peaks shown in CI 200 (see FIG. 2), i.e., peak 206 (representing intensity of DNC ions) and peak 208 (representing intensity of DNC-d8 ions), using an exemplary software program that is capable of analyzing LC-MS raw data. In an exemplary embodiment, with respect to FIG. 2, peaks 206 and 208 may have a peak area count equal to about 16,300,000 mm2 and 31,300,000 mm2, respectively. In an exemplary embodiment, with respect to step 108, calculating an exemplary area under an exemplary first peak and an exemplary area under an exemplary second peak may further include calculating a ratio of target analyte peak area to stable isotope-labeled analyte peak area.

In further detail with respect to step 110, step 110 may include detecting contamination of an exemplary food sample with an exemplary target analyte by comparing an exemplary area under an exemplary first peak with an exemplary area under an exemplary second peak. In an exemplary embodiment, detecting contamination of an exemplary food sample with an exemplary target analyte may include detecting contamination of an exemplary food sample with an exemplary target analyte by comparing an exemplary area under an exemplary first peak with an exemplary area under an exemplary second peak using an exemplary computing device. In an exemplary embodiment, detecting contamination of an exemplary food sample with an exemplary target analyte by comparing an exemplary area under an exemplary first peak with an exemplary area under an exemplary second peak may include one of detecting that an exemplary food sample may be contaminated with an exemplary target analyte if an exemplary area under an exemplary second peak is larger than an exemplary area under an exemplary first peak, or detecting that an exemplary food sample may not be contaminated with an exemplary target analyte if an exemplary area under an exemplary second peak is smaller than an exemplary area under an exemplary second peak. In an exemplary embodiment, detecting contamination of an exemplary food sample with an exemplary target analyte may include detecting that an exemplary food sample is contaminated with an exemplary target analyte if a ratio of target analyte peak area to stable isotope-labeled analyte peak area is more than 1 (>1), or detecting that an exemplary food sample is not contaminated with an exemplary target analyte if a ratio of target analyte peak area to stable isotope-labeled analyte peak area is less than 1 (<1). In an exemplary embodiment, an exemplary procedure similar to step 110 may be implemented to detect contamination of an exemplary food sample (e.g., chicken meat sample) with DNC based on IC 200 shown in FIG. 2. For example, in an exemplary embodiment, detecting contamination of an exemplary food sample (e.g., chicken meat sample) with DNC based on IC 200 may include comparing an exemplary area under peak 206 to an exemplary area under peak 208. As area under peak 206 (representing intensity of DNC ions) is smaller than area under peak 208 (representing intensity of DNC-d8 ions), an exemplary food sample (e.g., chicken meat sample) analyzed by an exemplary method similar to exemplary method 100 may be reported as “not contaminated with DNC”.

In an exemplary embodiment, exemplary method 100 may further include preparing a detection kit. In an exemplary embodiment, an exemplary detection kit may include a detection mixture. In an exemplary embodiment, an exemplary detection mixture may include a mixture of stable isotope-labeled contaminants formulated at concentrations corresponding to their respective MRLs. In an exemplary embodiment, an exemplary detection mixture may be used for spiking food matrices. In an exemplary embodiment, an exemplary detection mixture may be used for multi-residue screening of food and agricultural products to detect and quantify exemplary contaminants at levels aligned with established MRLs.

In an exemplary embodiment, an exemplary detection mixture may include a plurality of stable isotope-labeled analytes analogous to a plurality of target analytes. In an exemplary embodiment, one or more target analytes of an exemplary plurality of target analytes may be intended to be detected in an exemplary food sample. In an exemplary embodiment, an exemplary plurality of stable isotope-labeled analytes may include an exemplary stable isotope-labeled analyte and an exemplary plurality of target analytes may include an exemplary target analyte to be detected in an exemplary food sample. In an exemplary embodiment, using an exemplary detection mixture may allow for detection of more than one target analyte in an exemplary food sample simultaneously.

In an exemplary embodiment, a concentration of each stable isotope-labeled analyte of an exemplary plurality of stable isotope-labeled analytes in an exemplary detection mixture may correspond to a respective MRL of each target analyte of an exemplary plurality of target analytes. In an exemplary embodiment, spiking an exemplary food sample with an exemplary stable isotope-labeled analyte (step 102) may include adding an exemplary detection mixture to an exemplary food sample.

In an exemplary embodiment, preparing an exemplary detection mixture may include calculating an exemplary concentration of each stable isotope-labeled analyte of an exemplary plurality of stable isotope-labeled analytes equal to an exemplary Leq of each respective target analyte of an exemplary plurality of target analytes and mixing an exemplary plurality of stable isotope-labeled analytes at exemplary calculated concentrations together. In an exemplary embodiment, an exemplary Leq of each respective target analyte of an exemplary plurality of target analytes may be calculated using Equation 1 described herein above.

In an exemplary embodiment, mixing an exemplary plurality of stable isotope-labeled analytes at exemplary calculated concentrations together may include dissolving an exemplary plurality of stable isotope-labeled analytes at exemplary calculated concentrations in at least one of an aqueous solvent, an organic solvent, and combinations thereof. In an exemplary embodiment, mixing an exemplary plurality of stable isotope-labeled analytes at exemplary respective calculated concentrations together may include dissolving an exemplary plurality of stable isotope-labeled analytes at exemplary respective calculated concentrations in at least one of water, an alcohol, acetonitrile, and combinations thereof. In an exemplary embodiment, mixing an exemplary plurality of stable isotope-labeled analytes at exemplary respective calculated concentrations together may include dissolving an exemplary plurality of stable isotope-labeled analytes at exemplary respective calculated concentrations in methanol.

In an exemplary embodiment, preparing an exemplary detection mixture may further include removing at least one of an exemplary aqueous solvent, an exemplary organic solvent, and combinations thereof from an exemplary detection mixture. In an exemplary embodiment, removing at least one of an exemplary aqueous solvent, an exemplary organic solvent, and combinations thereof from an exemplary detection mixture may include separating at least one of an exemplary aqueous solvent, an exemplary organic solvent, and combinations thereof from an exemplary plurality of stable isotope-labeled analytes dissolved therein. In an exemplary embodiment, removing at least one of an exemplary aqueous solvent, an exemplary organic solvent, and combinations thereof from an exemplary detection mixture may include evaporating at least one of an exemplary aqueous solvent, an exemplary organic solvent, and combinations thereof from an exemplary detection mixture.

In an exemplary embodiment, exemplary method 100 may further include preventing whole of a food product associated with an exemplary food sample from distribution in markets if an exemplary food sample is detected to be contaminated with an exemplary target analyte in step 110. In an exemplary embodiment, an exemplary food sample may be sampled from an exemplary food product at the beginning of conducting method 100.

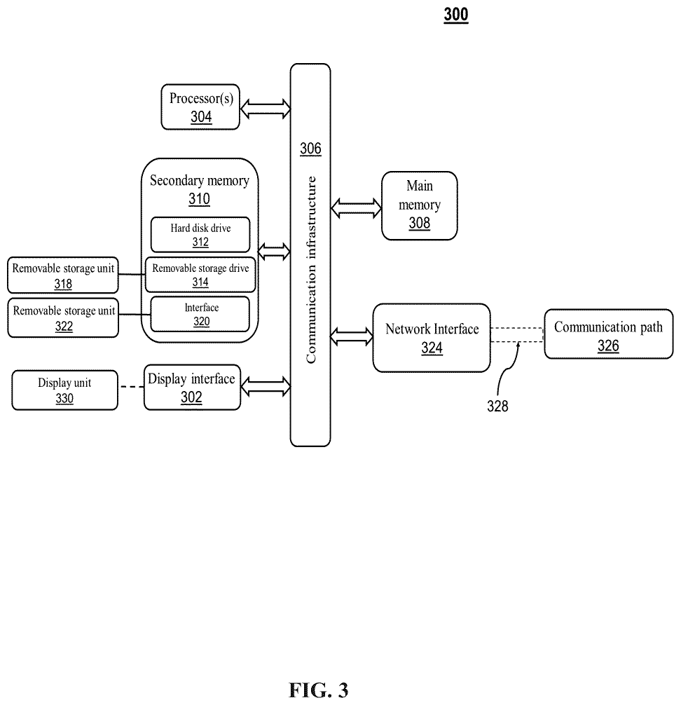

In an exemplary embodiment, total or parts of each of steps 106, 108, and 110 may be performed using exemplary one or more processors. In an exemplary embodiment, total or parts of each of steps 106, 108, and 110 may be performed using exemplary one or more processors of an exemplary computing device. FIG. 3 illustrates an example computer system 300 in which an embodiment of the present invention, or portions thereof, may be implemented as computer-readable code, consistent with exemplary embodiments of the present disclosure. For example, different steps of exemplary method 100 (e.g., steps 106, 108, and 110) may be implemented in computer system 300 using hardware, software, firmware, tangible computer readable media having instructions stored thereon, or a combination thereof and may be implemented in one or more computer systems or other processing systems. Hardware, software, or any combination of such may embody any of the modules and components in FIGS. 1-2.

If programmable logic is used, such logic may execute on a commercially available processing platform or a special purpose device. One ordinary skill in the art may appreciate that an embodiment of the disclosed subject matter may be practiced with various computer system configurations, including multi-core multiprocessor systems, minicomputers, mainframe computers, computers linked or clustered with distributed functions, as well as pervasive or miniature computers that may be embedded into virtually any device. For instance, a computing device having at least one processor device and a memory may be used to implement one or more exemplary embodiments described herein. A processor device may be a single processor, a plurality of processors, or combinations thereof. Processor devices may have one or more processor “cores.”

An embodiment of the disclosure may be described in terms of exemplary computer system 300. It may be apparent to a person skilled in the relevant art how to implement one or more exemplary embodiments using other computer systems and/or computer architectures. Although operations may be described as a sequential process, some of the operations may in fact be performed in parallel, concurrently, and/or in a distributed environment, and with program code stored locally or remotely for access by single or multiprocessor machines. In addition, in some embodiments the order of operations may be rearranged without departing from the spirit of the disclosed subject matter. Processor device 304 may be a special purpose (e.g., a graphical processing unit) or a general-purpose processor device. As will be appreciated by persons skilled in the relevant art, processor device 304 may also be a single processor in a multi-core/multiprocessor system, such system operating alone, or in a cluster of computing devices operating in a cluster or server farm. Processor device 304 may be connected to a communication infrastructure 306, for example, a bus, message queue, network, or multi-core message-passing scheme.

In an exemplary embodiment, computer system 300 may include a display interface 302, for example a video connector, to transfer data to a display unit 330, for example, a monitor. Computer system 300 may also include a main memory 308, for example, random access memory (RAM), and may also include a secondary memory 310. Secondary memory 310 may include, for example, a hard disk drive 312, and a removable storage drive 314. Removable storage drive 314 may include a floppy disk drive, a magnetic tape drive, an optical disk drive, a flash memory, or the like. Removable storage drive 314 may read from and/or write to a removable storage unit 318 in a well-known manner. Removable storage unit 318 may include a floppy disk, a magnetic tape, an optical disk, etc., which may be read by and written to by removable storage drive 314. As will be appreciated by persons skilled in the relevant art, removable storage unit 318 may include a computer usable storage medium having stored therein computer software and/or data. In alternative implementations, secondary memory 310 may include other similar means for allowing computer programs or other instructions to be loaded into computer system 300. Such means may include, for example, a removable storage unit 322 and interface 320. Examples of such means may include a program cartridge and cartridge interface (such as that found in video game devices), a removable memory chip (such as an EPROM, or PROM) and associated socket, and other removable storage units 322 and interfaces 320 which may allow software and data to be transferred from removable storage unit 322 to computer system 300.

Computer system 300 may also include a communications interface 324. Communications interface 324 may allow software and data to be transferred between computer system 300 and external devices. Communications interface 324 may include a modem, a network interface (such as an Ethernet card), a communications port, a PCMCIA slot and card, or the like. Software and data transferred via communications interface 324 may be in the form of signals, which may be electronic, electromagnetic, optical, or other signals capable of being received by communications interface 324. These signals may be provided to communications interface 324 via a communications path 326. Communications path 326 carries signals and may be implemented using wire or cable, fiber optics, a phone line, a cellular phone link, an RF link or other communications channels. In an exemplary embodiment, “computer program medium” and “computer usable medium” may be used to generally refer to media such as removable storage unit 318, removable storage unit 322, and a hard disk installed in hard disk drive 312. Computer program medium and computer usable medium may also refer to memories, such as main memory 308 and secondary memory 310, which may be memory semiconductors (e.g. DRAMs, etc.).

Computer programs (also called computer control logic) may be stored in main memory 308 and/or secondary memory 310. Computer programs may also be received via communications interface 324. Such computer programs, when executed, enable computer system 300 to implement one or more exemplary embodiments discussed herein. In particular, the computer programs, when executed, enable processor device 304 to implement the exemplary operations implemented in method 100 illustrated in FIG. 1 discussed above. Accordingly, such computer programs represent controllers of computer system 300. Where an exemplary embodiment of method 100 is implemented using an exemplary software, an exemplary software may be stored in an exemplary computer program product and loaded into computer system 300 using removable storage drive 314, interface 320, and hard disk drive 312, or communications interface 324. Exemplary embodiments also may be directed to computer program products including software stored on any computer useable medium. Such software, when executed in one or more data processing device, causes a data processing device to operate as described herein. An exemplary embodiment may employ any computer useable or readable medium. Examples of computer useable mediums include, but are not limited to, primary storage devices (e.g., any type of random-access memory), secondary storage devices (e.g., hard drives, floppy disks, CD ROMS, ZIP disks, tapes, magnetic storage devices, and optical storage devices, MEMS, nanotechnological storage device, etc.).

### EXAMPLES

Hereinafter, one or more exemplary embodiments will be described in further detail with reference to examples. It will be obvious to a person having ordinary skill in the art that these examples may be for illustrative purposes only and are not to be interpreted to limit the scope of the present disclosure.

### Example 1: Developing a Mathematical Model to Calculate Final Concentration of Stable Isotope-Labeled Analyte

In this example, an exemplary mathematical model was developed in order to be used in an exemplary procedure similar to step 102 of exemplary method 100. Maximum residue limit (MRL) may be a maximum concentration of a residue that is legally allowed or recognized as acceptable in a food product. An exemplary food sample may be considered as contaminated when concentration of an exemplary contaminant (an exemplary target analyte) in an exemplary food sample exceeds MRL of an exemplary contaminant. With further reference to FIG. 1, exemplary method 100 may utilize an exemplary stable isotope-labeled analyte, in a concentration equivalent to MRL of an exemplary target analyte, as a reference value to determine contamination of an exemplary food sample with an exemplary target analyte. In spite that a stable isotope-labeled analyte is structurally similar to its analogous unlabeled analyte, introducing a same concentration/mass of stable isotope-labeled analyte and its unlabeled analogue may result in LC-MS chromatograms that differ in signal intensity and peak area count. In this example, a mathematical operation was developed to calculate a final concentration of an exemplary stable isotope-labeled analyte (to be added to an exemplary food sample), i.e., Leq, that is equivalent to MRL of an exemplary target analyte and may result in a same mass spectrometry ion plot with the same intensity and peak area count as an exemplary ion plot of an exemplary target analyte with a concentration equal to MRL.

In this example, a number of parameters were identified to cause dissimilarity between peak area count of an exemplary target analyte and its analogous exemplary stable isotope-labeled analyte. In an exemplary embodiment, exemplary parameters (set forth in Equation 1) may include difference in molecular weight of an exemplary target analyte and its analogous stable isotope-labeled analyte, percentage purity (w/w) of an exemplary target analyte, and percentage purity (w/w) of an exemplary stable isotope-labeled analyte. With respect to molecular weight difference, due to the fact that an exemplary stable isotope-labeled analyte is composed of Deuterium or Carbon-13 (instead of Hydrogen and Carbon-12), an exemplary stable isotope-labeled analyte may have a larger molecular weight than an exemplary target analyte. Thus, equal mass of an exemplary stable isotope-labeled analyte and an exemplary target analyte may include different number of moles. For instance, when molecular weight of an exemplary target analyte is about 420 and molecular weight of its analogous stable isotope-labeled analyte is about 426, injecting equal mass of an exemplary target analyte and its analogous stable isotope-labeled analyte may result in an IC plot with two peaks that may have different signal intensity and peak area count (due to having different molar amount). Table 1 below shows calculated final concentrations (Leq) of a plurality of stable isotope-labeled analytes (using Equation 1) that may be equivalent to MRL of their analogous target analyte.

TABLE 1
Parameters of Equation 1 for calculating final concentrations (Leq) of Dinitrocarbanilide-d8, Enrofloxacin-
d5-HCl, Ciprofloxacin-d8, Marbofloxacin-d8, Danofloxacin-d3-mesylate, Flumequine-13C3, Sulfamethazine-d4,
and Spiramycin I-d3, consistent with one or more exemplary embodiments of the present disclosure.

| | | | | Molecular | | | |
|---|---|---|---|---|---|---|---|
| | Molecular | Purity of | Isotope- | Weight of | Purity of | | |
| | Weight of | Target | Labeled | Isotope- | Isotope- | MRL | |
| Target Analyte | [symbol] | analyt [symbol] | Analyte | [symbol] | [symbol] | (ng/mL) | L_{eq} |
| Dinitrocarbanilide | 302.24 | 100 | Dinitrocarbanilide-d₈ | 310.29 | 99.90 | 200 | 205.5324 |
| Enrofloxacin | 359.39 | 99.99 | Enrofloxacin-d₅-HCl | 400.89 | 99.99 | 100 | 111.5473 |
| Ciprofloxacin | 331.34 | 99.75 | Ciprofloxacin-d₈ | 339.39 | 98.91 | 100 | 103.2994 |
| Marbofloxacin | 362.36 | 100 | Marbofloxacin-d₈ | 370.40 | 96.00 | 75 | 79.8584 |
| Danofloxacin | 357.38 | 99.60 | Danofloxacin-d₃- [symbol] | 456.51 | 98.00 | 30 | 38.9470 |
| Flumequine | 261.25 | 99.40 | Flumequin¹³C₃ | 264.23 | 98.00 | 50 | 51.2927 |
| Sulfamethazine | 278.33 | 99.70 | Sulfamethazine-d₄ | 282.35 | 98.00 | 100 | 103.2040 |
| Spiramycin | 843.053 | 93.20 | Spiramycin I-d₃ | 846.70 | 95.00 | 200 | 196.9126 |

[symbol]

indicates data missing or illegible when filed

### Example 2: Optimization of Mass Spectrometry Conditions for Detecting Chemical Contaminants in Food

In this example, exemplary mass spectrometry (MS) parameters including ionization state, precursor mass, DP (Delustering Potential), primary CE (Collision energy), product mass and optimal CE, EP (Entrance Potential) and CXP (Collision cell exit potential) were optimized. To optimize exemplary features mentioned above, each standard sample (including an exemplary target analyte and an exemplary stable isotope-labeled analyte) was prepared in form of an exemplary aqueous or organic solution with a concentration of about 1 ppm. Then, an exemplary standard sample solution may be injected into an inlet chamber of an exemplary MS to measure molecular mass of each exemplary standard sample and mass of an exemplary target analyte and an exemplary stable isotope-labeled analyte in an exemplary standard sample was quantified. Then, selected ion monitoring (SIM) mode of an exemplary MS was used to determine an ion chromatogram (IC) of exemplary target analyte ions and exemplary stable isotope-labeled analyte ions. In an exemplary embodiment, multiple reaction monitoring (MRM) mode of an exemplary MS may be used to optimize different conditions of an exemplary MS including CE, DP, EP and CXP. Table 2 below sets forth optimized MRM settings for analyzing dinitrocarbanilide, quinolones (enrofloxacin, ciprofloxacin, marbofloxacin, danofloxacin and flumequine), sulfamethazine and spiramycin using an exemplary MS.

TABLE 2
Optimized multiple reaction monitoring (MRM) settings for mass spectrometry analysis of dinitrocarbanilide,
quinolones (enrofloxacin, ciprofloxacin, marbofloxacin, danofloxacin and flumequine), sulfamethazine
and spiramycin, consistent with one or more exemplary embodiments of the present disclosure.

| | | Q3 | | | | | | | |
|---|---|---|---|---|---|---|---|---|---|
| Analyte | Q1 | quantification | confirmation | Ionization | CE | DP | CXP | EP | Time |
| Dinitrocarbanilide | 301 | 136.9 | 106.9 | Negative | 16.23 | 50 | 11.09 | 8 | 50 |
| Dinitrocarbanilide-d₈ | 308.9 | 141 | 111 | Negative | 19.56 | 50 | 12.13 | 7 | 50 |
| Enrofloxacin | 360.1 | 244.9 | 316 | Positive | 26.30 | 107 | 16.7 | 4 | 50 |
| | | | 342 | | | | | | |
| Enrofloxacin -d₅-HCl | 365.1 | 245 | 321.2 | Positive | 28.56 | 30.46 | 17.19 | 9.86 | 50 |
| | | | 347 | | | | | | |
| Ciprofloxacin | 332.1 | 288.1 | 314.1 | Positive | 27.88 | 74.95 | 13 | 10.46 | 50 |
| | | | 244.9 | | | | | | |
| Ciprofloxacin -d₈ | 340.2 | 296.1 | 322 | Positive | 31.78 | 76.13 | 14.89 | 10.73 | 50 |
| | | | 249 | | | | | | |
| Marbofloxacin | 363 | 319.9 | 345 | Positive | 30.08 | 93.05 | 8.04 | 11 | 50 |
| | | | 72.1 | | | | | | |
| Marbofloxacin-d₈ | 372.2 | 328.9 | 354 | Positive | 31 | 59.09 | 15.95 | 7.36 | 50 |
| | | | 78.9 | | | | | | |
| Danofloxacin | 358.2 | 314.1 | 340.1 | Positive | 31.72 | 85.97 | 15.95 | 12 | 50 |
| | | | 283.1 | | | | | | |
| Danofloxacin-d₃- | 361.2 | 317.1 | 343 | Positive | 28.98 | 78.26 | 27 | 12.88 | 50 |
| mesylate | | | 283 | | | | | | |
| Flumequine | 262.3 | 202 | 243.7 | Positive | 25.04 | 69.68 | 11.04 | 10 | 50 |
| Flumequine-¹³C₃ | 265.2 | 204.8 | 246.9 | Positive | 26.14 | 88.27 | 7.51 | 3.47 | 50 |
| Sulfamethazine | 279 | 123.9 | 186 | Positive | 29.59 | 66.82 | 11.39 | 7.77 | 50 |
| Sulfamethazine-d₄ | 283.4 | 123.9 | 185.9 | Positive | 37.86 | 65.27 | 6.04 | 14 | 50 |
| Spiramycin | 843.6 | 100.9 | 174 | Positive | 85.89 | 103.04 | 10.08 | 9 | 50 |
| Spiramycin I-d₃ | 846.4 | 101 | 174.1 | Positive | 41.19 | 105.41 | 10 | 8 | 50 |

Ionization conditions may be altered due to variations in instrument components such as GS1 (nebulizing gas), GS2 (drying gas), DP and temperature of ionizer chamber. In an exemplary embodiment, GS1 and GS2 was adjusted to about 25 psi and ion source temperature was adjusted to about 500° C. Table 3 below shows exemplary optimized parameters of MS ionization chamber for testing food contamination.

TABLE 3
Optimized ionization conditions for testing food
contamination, consistent with one or more exemplary
embodiments of the present disclosure.

| Ion Spray Voltage | 5500 V | |
|---|---|---|
| | (Positive Mode) | |
| | 4500 V | |
| | (Negative Mode) | |
| Vaporizer temperature | 500° | C. |
| Capillary temperature | 350° | C. |
| Curtain gas pressure | 10 | psi |
| Collision gas pressure | 15 | psi |
| Source gas 1 pressure | 25 | psi |
| Source gas 2 pressure | 25 | psi |

### Example 3: Optimization of Liquid Chromatography Conditions for Detecting Chemical Contaminants in Food

In this example, exemplary liquid chromatography (LC) parameters including type and length of the LC column, type of organic solvent in mobile phase, and type of mobile phase (gradient or isocratic) may be optimized to obtain an LC-MS signal with a reasonable retention time (RT) for each of an exemplary target analyte and an exemplary stable isotope-labeled analyte. Tables 4-5 below, respectively, set forth optimized LC parameters for detecting dinitrocarbanilide and quinolones in an exemplary food sample.

TABLE 4
Optimized liquid chromatography parameters, including components
of mobile phase and retention time, for detecting dinitrocarbanilide
in an exemplary food sample (e.g., chicken meat), consistent with
one or more exemplary embodiments of the present disclosure.

| | |
|---|---|
| Column chromatography: Kinetex ®-XB-C18 | Stationary |
| (4.6 mm × 150 mm, 3. 5 μm) | phase |
| Aqueous phase: deionized water containing | Mobile phase |
| 0.1% formic acid and 5 mM ammonium acetate | |
| Organic phase: methanol containing 0.1% | |
| formic acid and 5 mM ammonium acetate | |

Isocratic program used

| Retention | Flow rate | Aqueous | Organic |
|---|---|---|---|
| time (min) | (mL/min) | phase (%) | phase (%) |
| 12 | 0.4 | 50 | 50 |
| 9.2 | 0.4 | 25 | 75 |

TABLE 4
Optimized liquid chromatography parameters, including components
of mobile phase and program conditions, for detecting quinolones
in an exemplary food sample (e.g., milk), consistent with one
or more exemplary embodiments of the present disclosure.

| | |
|---|---|
| Column chromatography: Kinetex ®-XB-C18 | Stationary |
| (4.6 mm × 150 mm, 3. 5 μm) | phase |
| Aqueous phase: deionized water containing | Mobile phase |
| 0.1% formic acid and 5 mM ammonium format | |
| Organic phase: methanol containing 0.1% | |
| formic acid and 5 mM ammonium format | |

Gradient Program

| Retention | Flow rate | Aqueous | Organic |
|---|---|---|---|
| time (min) | (mL/min) | phase (%) | phase (%) |
| 0.4 | 95% | 5% | 0 |
| 0.4 | 95% | 5% | 0.01 |
| 0.4 | 80% | 20% | 3 |
| 0.4 | 65% | 35% | 6 |
| 0.4 | 50% | 50% | 9 |
| 0.4 | 35% | 65% | 11 |
| 0.4 | 20% | 80% | 13 |
| 0.4 | 5% | 95% | 15 |
| 0.4 | 5% | 95% | 17 |
| 0.4 | 95% | 5% | 20 |

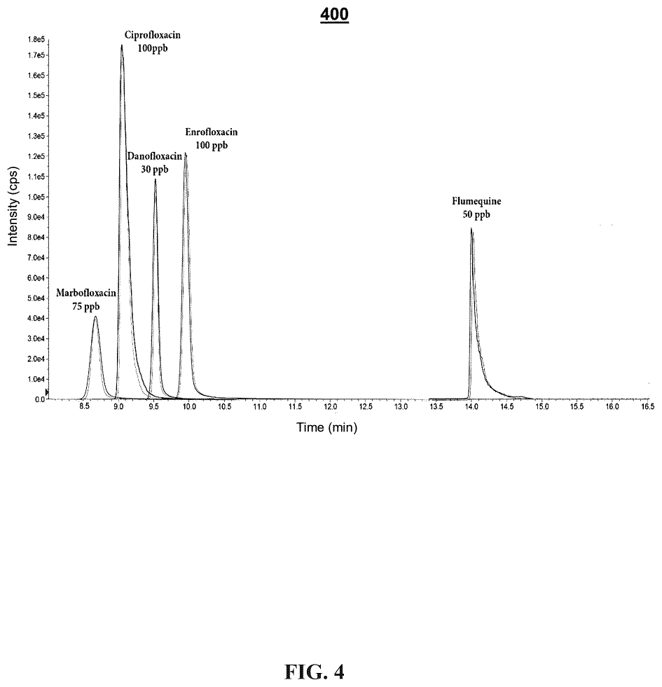

In an exemplary embodiment, the obtained chromatogram using gradient program set forth in Table 4 is shown in FIG. 4. FIG. 4 illustrates exemplary SIC plot 400 obtained from LC-MS analysis of a milk sample spiked with a mixture of target quinolones (enrofloxacin, ciprofloxacin, danofloxacin, marbofloxacin and flumequine) and a mixture of stable isotope-labeled quinolones analogous to target quinolones, consistent with one or more exemplary embodiments of the present disclosure. In an exemplary embodiment, exemplary SIC plot 400 may be formed by an exemplary method similar to step 106 of exemplary method 100. In further detail with respect to IC plot 400, overlapping of each target quinolone peak with its analogous isotope-labeled quinolone (set forth in Table 1 and Table 2) may further validate applicability of Equation 1 for analyzing food contamination without needing to create calibration curve. Furthermore, in this example, exemplary LC parameters were optimized for simultaneous detection of sulfamethazine and spiramycin in an exemplary food sample. Table 5 below sets forth optimized LC parameters for detecting sulfamethazine and spiramycin in an exemplary food sample (e.g., milk).

TABLE 5
Optimized liquid chromatography parameters, including
components of mobile phase and program conditions, for
detecting sulfamethazine and spiramycin in an exemplary
food sample (e.g., milk), consistent with one or more
exemplary embodiments of the present disclosure.

| | |
|---|---|
| Column chromatography: Kinetex ®-XB-C18 | Stationary |
| (4.6 mm × 150 mm, 3.5 μm) | phase |
| Aqueous phase: deionized water containing | Mobile phase |
| 0.1% formic acid | |
| Organic phase: acetonitrile containing | |
| 0.1% formic acid | |

Gradient program

| Retention | Flow rate | Aqueous | Organic |
|---|---|---|---|
| time (min) | (mL/min) | phase (%) | phase (%) |
| 0.4 | 95% | 5% | 0 |
| 0.4 | 70% | 30% | 2 |
| 0.4 | 30% | 70% | 6 |
| 0.4 | 90% | 10% | 8 |
| 0.4 | 95% | 5% | 10 |

### Example 4: Developing a Detection Kit for Screening Dinitrocarbanilide

In this example, an exemplary detection kit was developed for screening dinitrocarbanilide (DNC) in chicken meat using ready-to-use solutions. In an exemplary embodiment, dinitrocarbanilide-d₈ (DNC-d₈) with a concentration (L_{eq}) equivalent to MRL of DNC in chicken meat was calculated using Equation 1 (e.g., using computer system 300) and added to an exemplary laboratory container (e.g., vial, falcon, microtube, etc.). An exemplary kit for detecting DNC may include at least one detection mixture (e.g., 1000 detection mixture) for analyzing an exemplary chicken meat (each detection mixture may be used for testing a certain unit weight (e.g., 1 g) of chicken meat sample). To prepare an exemplary detection mixture for screening DNC in an exemplary chicken meat sample, first an exemplary concentration (L_{eq}) of DNC-d₈ (in ppm or ppb) equivalent to MRL of DNC was calculated using Equation 1 to be about 205.53 ng/g of an exemplary chicken meat sample. In an exemplary embodiment, an exemplary detection mixture of an exemplary DNC-screening kit may be prepared for testing about 5 g of a sample chicken meat in each test. In an exemplary embodiment, to test 5 g of a sample chicken meat, about 1027.6 ng of DNC-d₈ may be needed. Thus, to prepare a detection mixture for testing 1000 samples, about 1.03 mg of DNC-d₈ may be weighed and added to a one or more vials.

For example, to test a sample of chicken meat, a user may add about 5 mL methanol to an exemplary vial of DNC-d₈ (containing about 1.03 mg of DNC-d₈) and shake an exemplary vial to obtain a clear solution. An exemplary formed methanolic solution may then be transferred to a 50 mL volumetric flask and made up to a volume of about 50 mL by adding an about 45 mL methanol to 50 mL volumetric flask. To test an exemplary chicken meat sample, about 50 μL of an exemplary prepared DNC-d₈ solution (per each gram of chicken meat) may be added to an exemplary chicken meat sample (e.g., about 250 u L of DNC-d₈ solution may be added to about 5 g of an exemplary chicken sample). Subsequently, an exemplary acetonitrile-based extraction procedure (similar to an exemplary procedure set forth in step 104 of exemplary method 100) may be used to extract an exemplary DNC-d₈ and DNC from an exemplary spiked chicken sample. Extracted DNC-d₈ and DNC may then be injected into an inlet source of an exemplary LC-MS to obtain an exemplary IC plot using an exemplary procedure similar to step 106 of exemplary 100.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for detecting contamination of a food sample with a target analyte, the method comprising:
selecting a stable isotope-labeled analyte analogous to the target analyte;
spiking the food sample with the stable isotope-labeled analyte at a concentration equivalent to maximum residue limit (MRL) level of the target analyte being permissible to be present in the food sample, spiking the food sample with the stable isotope-labeled analyte comprises adding the stable isotope-labeled analyte with a final concentration equal to L_{eq} to the food sample,
wherein L_{eq} is defined by the following operation:
$${L}_{eq} = {W}_{N} \times \frac{MW ( L )}{MW ( N )} \times \frac{purity ( N )}{purity ( L )} ,$$
wherein L_{eq} is the final concentration of the stable isotope-labeled analyte in the spiked food sample, W_{N} is a maximum residue limit (MRL) of the target analyte, MW(L) is a molecular weight of the stable isotope-labeled analyte, MW(N) is a molecular weight of the target analyte, purity(N) is a percentage purity (w/w) of the target analyte, and purity(L) is a percentage purity (w/w) of the stable isotope-labeled analyte;
extracting the stable isotope-labeled analyte and the target analyte from the spiked food sample;
generating an ion chromatogram (IC) by introducing the extracted stable isotope-labeled and target analyte into a liquid chromatography-mass spectrometer (LC-MS), wherein the IC comprises a first peak representing an amount of the stable isotope-labeled analyte in the spiked food sample and a second peak representing an amount of the target analyte in the spiked food sample;
calculating, utilizing one or more processors, an area under the first peak and an area under the second peak; and
detecting contamination of the food sample with the target analyte by detecting the amount of the target analyte in the spiked food sample exceeds the MRL of the target analyte, detecting the contamination of the food sample with the target analyte comparing the area under the first peak with the area under the second peak, detecting the contamination of the food sample with the target analyte comprising:
detecting the food sample is contaminated with the target analyte if the area under the second peak is larger than the area under the first peak; or
detecting the food sample is not contaminated with the target analyte if the area under the second peak is smaller than the area under the second peak.

2. A method for detecting contamination of a food sample with a target analyte, the method comprising:
spiking the food sample with a stable isotope-labeled analyte analogous to the target analyte at a level equivalent to a maximum residue limit (MRL) of the target analyte being permissible to be present in the food sample, spiking the food sample with the stable isotope-labeled analyte at the level equivalent to the MRL of the target analyte comprising adding an amount of the stable isotope-labeled analyte with a final concentration to the food sample equivalent to the MRL of the target analyte;
extracting the stable isotope-labeled analyte and the target analyte from the spiked food sample;
generating an ion chromatogram (IC) by introducing the extracted stable isotope-labeled and target analyte into a liquid chromatography-mass spectrometer (LC-MS), the IC comprising a first peak representing an amount of the stable isotope-labeled analyte in the spiked food sample and a second peak representing an amount of the target analyte in the spiked food sample;
calculating, utilizing one or more processors, an area under the first peak and an area under the second peak; and
detecting, utilizing one or more processors, contamination of the food sample with the target analyte by detecting the amount of the target analyte in the spiked food sample exceeds the MRL of the target analyte, detecting the contamination of the food sample with the target analyte comparing the area under the first peak with the area under the second peak, detecting the contamination of the food sample with the target analyte comprising:
detecting the food sample is contaminated with the target analyte if the area under the second peak is larger than the area under the first peak; or
detecting the food sample is not contaminated with the target analyte if the area under the second peak is smaller than the area under the second peak.

3. The method of claim 2, wherein spiking the food sample with the stable isotope-labeled analyte at the level equivalent to the MRL of the target analyte comprises adding the stable isotope-labeled analyte with a final concentration equal to L_{eq} to the food sample,
wherein L_{eq} is defined by the following operation:
$${L}_{eq} = {W}_{N} \times \frac{MW ( L )}{MW ( N )} \times \frac{purity ( N )}{purity ( L )} ,$$
wherein L_{eq} is the final concentration of the stable isotope-labeled analyte in the spiked food sample, W_{N} is a maximum residue limit (MRL) of the target analyte, MW(L) is molecular weight of the stable isotope-labeled analyte, MW(N) is molecular weight of the target analyte, purity(N) is a percentage purity (w/w) of the target analyte, and purity(L) is a percentage purity (w/w) of the stable isotope-labeled analyte.

4. The method of claim 3, wherein W_{N} comprises the MRL of the target analyte in parts per million (ppm) of the food sample.

5. The method of claim 3, wherein W_{N} comprises the MRL of the target analyte in parts per billion (ppb) of the food sample.

6. The method of claim 3, wherein spiking the food sample with the stable isotope-labeled analyte at the level equivalent to the MRL of the target analyte comprises:
selecting the stable isotope-labeled analyte;
calculating, utilizing one or more processors, the L_{eq}; and
adding the stable isotope-labeled analyte with the final concentration equal to L_{eq} to the food sample.

7. The method of claim 2, wherein extracting the stable isotope-labeled analyte and the target analyte from the spiked food sample comprises extracting the stable isotope-labeled analyte and the target analyte from the spiked food sample through at least one of a solid-liquid extraction process, a liquid-liquid extraction process, a solid-phase extraction process, and combinations thereof.

8. The method of claim 2, wherein detecting the contamination of the food sample with the target analyte comprises detecting contamination of the food sample with a chemical target analyte.

9. The method of claim 8, wherein detecting the contamination of the food sample with the target analyte comprises detecting contamination of the food sample with at least a pesticide compound, an antibiotic compound, a drug, a toxin, a heavy metal, a persistent Organic Pollutant (POP), Brominated Fire Retardants (BFR), a phthalate, a dioxins, halogenated compounds, hormones, and combinations thereof.

10. The method of claim 8, wherein detecting the contamination of the food sample with the target analyte comprises detecting contamination of the food sample with at least one of Dinitrocarbanilide, Enrofloxacin-HCl, Ciprofloxacin, Marbofloxacin, Danofloxacin-mesylate, Flumequine, Sulfamethazine, and Spiramycin I, and combinations thereof.

11. The method of claim 2, wherein detecting the contamination of the food sample with the target analyte comprises detecting the contamination of a sample of at least one of baked goods, baking mixes, flours, coffee, tea, milk, breakfast cereals, fats and oils, condiments, relishes, fresh fruits, fruit juices, herbs, spices, fresh vegetables, meat products, jams, jellies, snack foods, soups, sugars, water, rice, corn, wheat, coffee grounds, tea leaves, food additives, chemicals used in food production, dyes, color additives, vitamins, dietary supplements, medicines, cosmetics, and combinations thereof.

12. The method of claim 2, wherein generating the IC comprises:
injecting the extracted stable isotope-labeled and target analyte into an inlet chamber of the LC-MS using a syringe pump; and
plotting, utilizing one or more processors, the IC associated with the injected extracted stable isotope-labeled and target analyte, the IC comprising peak intensities of the injected extracted stable isotope-labeled and target analyte versus retention time (RT) of the injected extracted stable isotope-labeled and target analyte passing through a chromatography column of the LC-MS.

13. The method of claim 2, wherein comparing the area under the first peak with the area under the second peak comprises:
calculating, utilizing one or more processors, a ratio of target analyte peak area to stable isotope-labeled analyte peak area by dividing the area under the second peak to the area under the first peak; and
detecting, utilizing one or more processors, contamination status of the food sample, comprising:
detecting that the food sample is contaminated with the target analyte if the ratio of the target analyte peak area to the stable isotope-labeled analyte peak area is more than 1 (>1); or
detecting that the food sample is not contaminated with the target analyte if the ratio of the target analyte peak area to the stable isotope-labeled analyte peak area is less than 1 (<1).

14. The method of claim 2, further comprising preventing whole of food product associated with the food sample from distribution in markets responsive to detecting the food sample is contaminated with the target analyte.

15. The method of claim 3, further comprising:
preparing a detection mixture comprising a plurality of stable isotope-labeled analytes analogous to a plurality of target analytes, a concentration of each stable isotope-labeled analyte of the plurality of stable isotope-labeled analytes corresponding to a respective MRL of each target analyte of the plurality of target analytes, the plurality of stable isotope-labeled analytes comprising the stable isotope-labeled analyte and the plurality of target analytes comprising the target analyte.

16. The method of claim 15, wherein spiking the food sample with the stable isotope-labeled analyte comprises adding the detection mixture to the food sample.

17. The method of claim 15, wherein preparing the detection mixture comprises:
calculating the concentration of each stable isotope-labeled analyte of the plurality of stable isotope-labeled analytes equal to the L_{eq} of a respective target analyte of the plurality of target analytes; and
mixing the plurality of stable isotope-labeled analytes at the respective calculated concentrations together.

18. The method of claim 17, wherein mixing the plurality of stable isotope-labeled analytes at the respective calculated concentrations together comprises dissolving the plurality of stable isotope-labeled analytes at the respective calculated concentrations in at least one of an aqueous solvent, an organic solvent, and combinations thereof.

19. The method of claim 18, wherein mixing the plurality of stable isotope-labeled analytes at the respective calculated concentrations together comprises dissolving the plurality of stable isotope-labeled analytes at the respective calculated concentrations in at least one of water, an alcohol, acetonitrile, and combinations thereof.

20. The method of claim 17, wherein preparing the detection mixture further comprises removing the at least one of the aqueous solvent, the organic solvent, and combinations thereof from the plurality of stable isotope-labeled analytes dissolved therein.
